SEQUENCE LISTING (9 sequences)

SEQ ID NO: 1 - DNA, 22 bp, Artificial Sequence
  Description of Artificial Sequence Synthetic oligonucleotide
ctgctgacgatgagtcctgagt

SEQ ID NO: 2 - DNA, 11 bp, Artificial Sequence
  Description of Artificial Sequence Synthetic oligonucleotide
cgactcaggac

SEQ ID NO: 3 - DNA, 38 bp, Artificial Sequence
  Description of Artificial Sequence Synthetic oligonucleotide
ctgctgacgatgagtcctgagtcggnnnnnnnnnnnnn

SEQ ID NO: 4 - DNA, 38 bp, Artificial Sequence
  Description of Artificial Sequence Synthetic oligonucleotide
nnnnnnnnnnnnnccgactcaggactcatcgtcagcag

SEQ ID NO: 5 - DNA, 24 bp, Artificial Sequence
  Description of Artificial Sequence Synthetic oligonucleotide
gacgactgctactcaggactcagc

SEQ ID NO: 6 - DNA, 25 bp, Artificial Sequence
  Description of Artificial Sequence Synthetic oligonucleotide
ctgctgacgatgagtcctgagtcgg

SEQ ID NO: 7 - DNA, 46 bp, Artificial Sequence
  Description of Artificial Sequence Synthetic oligonucleotide
ctgctgacgatgagtcctgagtcgactcaggactcatcgtcagcag

SEQ ID NO: 8 - DNA, 46 bp, Artificial Sequence
  Description of Artificial Sequence Synthetic oligonucleotide
ctgctgacgatgagtcctgagtcggnnnnnnnnnnnnnnnnnnnnn

SEQ ID NO: 9 - DNA, 46 bp, Artificial Sequence
  Description of Artificial Sequence Synthetic oligonucleotide
nnnnnnnnnnnnnnnnnnnnnccgactcaggactcatcgtcagcag

(19) United States
(12) Patent Application Publication
(10) Pub. No.: US 2006/0183115 A1
(43) Pub. Date: Aug. 17, 2006

(54) Assays for dna methylation changes

(75) Inventor: Zunde Wang, Santa Barbara, CA (US)

Correspondence Address: FOLEY & LARDNER LLP, P.O. BOX 80278, SAN DIEGO, CA, 92138-0278, US

(73) Assignee: EpiGenX Pharmaceuticals

(21) Appl. No.: 10/489,509

(22) PCT Filed: Sep. 26, 2002
(86) PCT No.: PCT/US02/30977

§ 371 Date: Oct. 14, 2004

Related U.S. Application Data
(60) Provisional application No. 60/326,045, filed on Sep. 26, 2001.

(51) Int. Cl. C12Q 1/68; C12P 19/34

(52) U.S. Cl. 435/6; 435/91.2

(57) ABSTRACT

A new technique to determine the extent of DNA methylation entails generating DNA fragments of a test sample by cleaving at methylation sites that are not methylated while sparing methylation sites in the DNA that are methylated. This approach provides enhanced sensitivity to differences of even a single methylated cytosine, within or outside of a CpG island, and yet it can be employed to ascertain the methylation status of a region of DNA comprising a CpG island, a DNA region comprising one or more CpG-containing islands, or even a large variety of DNA regions.

### FIELD OF THE INVENTION

The present invention relates to the detection of a modified nucleotide sequence and, more specifically, to the detection of nucleic acid base methylation of a nucleotide sequence.

### BACKGROUND OF THE INVENTION

The methylation of DNA is thought to have important effects in the regulation of gene expression in eukaryotes, having epigenetic and mutagenic effects on various cellular activities such as differential gene expression, cell differentiation, chromatin inactivation, genomic imprinting, and carcinogenesis. Gonzalgo & Jones, Mutat. Res. 386:107-118 (1997). DNA methylation is also known to have a role in regulating gene expression during cellular development. Huang et al., Human Molecular Genetics 8: 459-470 (1999). In mammals, DNA methylation usually occurs at cytosines located 5′ of guanines, known as CpG dinucleotides. Many CpGs are clustered at/near regulatory regions of genes, which are called CpG islands. DNA methylation or hypermethylation within CpG islands is thought to be especially important in regulating gene expression. A significant number of genes are associated with CpG islands and CpG island hypermethylation has been observed in more than 90 gene promoters. CpG island hypermethylation is associated with the epigenetic inactivation of tumor suppressor or growth regulatory genes in cancer.

Because methylation of DNA often is reflective of disease status, accurate, sensitive, and efficient methods of detecting changes in methylation patterns would be highly desirable. The existing methods of detecting changes in methylation patterns frequently require laborious steps, such as subtractive hybridization and analysis using Southern blots. Existing methods that depend on reacting unmethylated cytosines with chemical reagents like sodium bisulfite can damage templates and alter base composition, causing problems with a subsequent PCR amplification. Existing methods have other weaknesses such as an inability to detect methylation changes for the entire genome with a high degree of sensitivity. Accordingly, improved methods with higher throughput and increased accuracy are needed for detecting methylation of DNA.

### SUMMARY OF THE INVENTION

The present invention provides methods for detecting whether the extent of methylation of one or more regions of DNA in a test sample is different from that of a control.

In one aspect, the invention comprises generating DNA fragments from at least one test sample of DNA by cleaving methylation sites that are not methylated while sparing methylation sites in the DNA that are methylated, ligating an oligonucleotide-linker to the ends of the DNA fragments, amplifying the DNA by initiating amplification from the linker, hybridizing the amplified DNA to one or more polynucleotides immobilized on a solid support, the polynucleotides complementary to one or more regions of DNA in the test sample, and comparing the amplified DNA from the test sample that hybridizes to the immobilized polynucleotides versus that of a control. In an exemplary embodiment the DNA fragments may be labeled with a detectable moiety.

In another aspect, the invention comprises generating DNA fragments from at least one test sample of DNA by cleaving methylation sites that are not methylated while sparing methylation sites in the DNA that are methylated, separating the fragments of appropriate sizes, ligating an oligonucleotide-linker that is labeled with a detectable moiety to the ends of the DNA fragments, hybridizing the amplified DNA to one or more polynucleotides immobilized on a solid support, the polynucleotides complementary to one or more regions of DNA in the test sample, and comparing the amount of the detectable moiety associated with the polynucleotide for the test sample versus a control.

In another aspect, the present invention uses the methods described above to determine if an individual has a state of disease associated with an abnormal extent of methylation of one or more regions of DNA in the individual. The above aspects of the invention have many different embodiments which will be addressed in detail below.

### BRIEF DESCRIPTION OF THE DRAWINGS

FIG. 1 is a schematic illustration of one embodiment of the inventive methodology. Region A and B represent segments of genomic DNA present in DNA from a test sample and a control sample. Vertical lines indicate the location of methylation sites in the DNA. Vertical lines topped with an “M” indicate that the methylation site is methylated. Thus, as depicted, both DNA regions are not methylated in the control sample while region B is methylated in the test sample. Selective amplification of regions bordered by non-methylated methylation sites is achieved by digesting the DNA into fragments with a methylation sensitive restriction enzyme, ligating the fragments with an oligonucleotide linker, and amplifying the linker ligated fragments with a linker primer. The amplified product from the test and control samples are differentially labeled as shown with Cy3 and Cy5 cyanine dye. Labeled amplicons from the two samples are combined and hybridized to an array with fixed probes for region A and B. The microarray is scanned at the excitation wavelength of 532 nm and 635 nm. Dual signals for probe A indicate that region A in the test sample is not methylated while a single signal for probe B indicates that region B in the test sample is methylated.

FIG. 2 illustrates the use of unphosphorylated linkers in a scheme for amplifying fragments generated by cleavage with a methylation sensitive restriction enzyme. Vertical bars represent matched base pairs. Panel A shows a double stranded HpaII (C′CGG) linker with a top strand (SEQ ID NO: 1) and a short bottom strand with no 5′-phosphate (SEQ ID NO: 2). Panel B shows a DNA fragment following HpaII restriction. Panel C shows the DNA formed following ligation of the unphosphorylated linker to the HpaII fragment. As seen, the short bottom strand of the linker lacking a 5′ phosphate (SEQ ID NO: 2) is not ligated, leaving a nick, while the top strand is ligated to the 5′ phosphate of the fragment (resulting in SEQ ID NO: 3). Addition of amplification reactants (polymerase and dNTPs) to the ligated fragments followed by a short incubation at 72° C. causes the un-ligated lower strand of the linker to be replaced by a newly synthesized region (see bold font) (SEQ ID NO: 4). Subsequent temperature cycling leads to amplification by priming with unused unphosphorylated linker top strand.

FIG. 3 illustrates the principle of amplicon preparation for interrogating rare methylation sites by cleaving with both a methylation sensitive (“MS”) and a methylation insensitive (“MIS”) restriction enzyme. As amplification is desired only for fragments involving an MS restriction (MS/MS and MS/MIS). Panel A indicates that two types of linkers are used: a phosphorylated linker for MS cleavage sites and an unphosphorylated linker for MIS cleavage sites. Panel B shows that following restriction with an MS and MIS enzyme, the lower strand of the MIS linker cannot be ligated because of the missing 5′ phosphate. Thus, a nick is present between the short bottom strand of the linker and the fragment. This non-covalently associated lower strand of the linker DNA is unstable and cannot support primer directed amplification. Thus, MIS/MIS fragments with non-covalently attached linker DNA at both ends of the fragments are not amplifiable while fragments generated by MS only (MS/MS) or by MS at one end and MIS at the other end (MS/MIS) can be amplified.

FIG. 4 illustrates how a linker primer can be designed to prevent amplification of linker dimers. Vertical bars represent matched base pairs. Panel A shows a double stranded (SEQ ID NO: 1 and 5) HpaII (C′CGG) linker with a 5′-phosphate. Panel B shows a DNA fragment following HpaII enzyme restriction. Panel C shows a linker primer (SEQ ID NO: 6) for avoiding amplification of linker dimers. Panel D1 depicts a linker dimer (SEQ ID NO:7) formed from the linker in panel A. Panel D2 shows a mismatched base (guanine) at the 3′ end of the linker primer (SEQ ID NO: 6) when hybridized to the bottom strand of the linker dimer (SEQ ID NO:7). The presence of a mismatched 3′ end nucleotide prohibits the primer from directing amplification. Panel E1 shows linker dimer ligated to the HpaII fragment (resulting in SEQ ID NO: 8 and 9). Panel E2 shows that there is no mismatched base at the 3′ end of the linker primer (SEQ ID NO:6) when hybridized to the bottom strand of the ligated fragment (SEQ ID NO:9). The absence of a mismatched 3′ base in the linker primer provides for primer directed amplification. Thus, amplification of linker dimers is avoided by extending the linker primer at its 3′ end until a 3′ base incompatibility is obtained between the primer and the primer dimer (in this example only a one base extension is required) while maintaining 3′ end compatibility between the linker primer and the DNA fragment.

FIG. 5 demonstrates that the inventive methylation detection method can identify strain specific methylation of a transgene. Panel A is a Southern blot of mouse DNA from transgenic strains D and B hybridized to a labeled probe for the transgene. The blot shows that the transgene is methylated in strain B but not in strain D (the increased molecular weight of the methylated (“meth”) transgene versus the unmethylated (“um”) transgene results from preparing the Southern using a methylation sensitive restriction enzyme). Panel B shows that when amplicons are prepared from strain D and B as described in Example 1, amplified transgene can be detected only from strain D (the strain that does not methylate the transgene). Gel shown panel B was prepared by electrophoresis of transgene specific PCR of the amplicons. Arrow indicates the position of amplified transgene. Panel C shows the amounts of transgene and an unmethylated control gene present in amplicons prepared from strain D and B as described in panel B. The amplicons from strain D were labeled with Cy5 while the amplicons from strain B were labeled with Cy3. The two amplicons were combined and hybridized to an oligonucleotide microarray containing an oligonucleotide for the transgene and one for an unmethylated control gene (both in triplicate). The table shows the amount of Cy3 and Cy5 dye hybridized to the transgene oligonucleotide and the control gene oligonucleotide. The observed reduction in relative Cy3 signal indicates that the transgene in strain B (labeled with Cy3) is methylated as compared to that in strain D. In contrast, the relative similarity in Cy3 and Cy5 signal for the control gene indicates that this gene is similarly methylated in the two strains of mice.

### DETAILED DESCRIPTION OF THE INVENTION

As noted, methylation of DNA in mammals occurs at cytosines, including cytosines in CpG sites, by addition of a methyl group at the pyrimidine ring of the cytosine. A new approach developed herein for determining the extent of methylation change of one or more regions of DNA is provided. The inventive methodology can be used to determine whether the extent of methylation of a DNA region in one sample differs from that of a control.

In one aspect, the approach of the present invention provides an enhanced sensitivity to differences of even a single methylated cytosine, within or outside of a CpG island, and yet it can be employed to ascertain the methylation status of a region of DNA comprising one or more CpG islands, one or more regions of DNA comprising one or more CpG containing islands, or even a large variety of DNA regions. The latter may entail determining the methylation status of between about 10,000 to 50,000 or more DNA segments of the genome of a single individual. For example, one could use the method to interrogate the extent of methylation present at all known CpG rich sites (about 45,000 presently known). In another aspect, hundreds or more samples can be analyzed everyday in a standard molecular biology laboratory and a microarray facility. Thus, the present invention allows for rapid and relatively inexpensive interrogation of the methylation status, genome-wide, for many individuals.

In one aspect, the present invention comprises generating DNA fragments from a sample of DNA by cleaving methylation sites in the DNA that are not methylated while sparing methylation sites in the DNA that are methylated. Oligonucleotide-linkers are ligated to the ends of the DNA fragments. In some embodiments, the linkers are labeled with a detectable moiety. In other embodiments, the DNA is amplified by initiating amplification from the oligonucleotide-linkers. The DNA fragments are analyzed by hybridization to one or more polynucleotides immobilized on a solid support. Finally, the amount of DNA associated with the solid support for the test sample DNA is compared to that of a control to determine whether the extent of methylation of the regions of DNA sought to be evaluated is different between the test sample and the control. In some embodiments, an actual control sample of DNA is used and the test and DNA fragments may be mixed together before hybridization to the immobilized polynucleotides. A discussion of the various steps of these methods as well as additional embodiments based on modifications and/or additions to the procedure follows.

DNA Fragmentation

DNA fragments can be generated from DNA purified from a cell, tissue or body fluid. Techniques for isolating DNA from such sources are well known to those skilled in the art. See, for example, Sambrook et al., MOLECULAR CLONING: A LABORATORY MANUAL, Vols. 1-3 (Cold Spring Harbor Laboratory Press, 1989). Highly pure DNA is easy to prepare and is preferred, but lower degrees of purity may be used provided the DNA can be cleaved and processed as required in subsequent steps.

DNA fragments are generated by cleavage at methylation sites that are not methylated while sparing methylation sites in the DNA that are methylated. In this description, the phrase “methylation sites” refers to sites in the DNA that potentially could be methylated in a cell. Such sites contain a cytosine, which is found often (but not exclusively) in a CpG context. A preferred manner for cleaving DNA fragments at methylation sites that are not methylated, while sparing sites in the DNA that are methylated, is to cleave the DNA with one or more methylation-sensitive agents. A “methylation-sensitive” agent is one that normally cleaves DNA at a methylation site when the site is unmethylated, but does not cleave the site, or cleaves it at low efficiency, when the site is methylated. A methylation-sensitive agent preferably recognizes and cleaves only at methylation sites in the DNA when they are not methylated. Methylation sensitive agents that recognize methylation sites involving methylation of adenine or cytosine nucleotides in bacterial DNA or cytosine nucleotides in CpG, CpNpG and CCWGG sites in plant or mammal DNA can be used.

A preferred methylation sensitive agent is a methylation sensitive restriction enzyme. Such enzymes and details for their use are commercially available from, for example, New England BioLabs, ProMega Biochem and Boehringer-Mannheim, among others. Techniques for identifying other methylation-sensitive restriction enzymes are known to the skilled artisan. Sambrook et al., supra, (1989) provides a general description of methods for using restriction enzymes and other enzymes, for instance.

Illustrative methylation-sensitive restriction enzymes, suitable for use in the present invention for analyzing methylation changes in CpG sites, include HpaII (C′CGG), AciI (C′CGC), HinP1I (G′CGC), HpyCH4IV (A′CGT), EagI (C′GGCCG), NgoMIV (G′CCGGC), KasI (G′GCGCC), SmaI (CCC′GGG), BstUI (CG′CG), BspDI (AT′CGTA), BstBI (TT′CGAA), SalI (G′TCGAG), and XhoI (C′TCGAG). In accordance with the present invention, cleavage may be achieved by more than one agent, used separately or in combination. Enzymes such as EagI, NgoMIV, KasI and SmaI are especially useful because they are located mainly in CpG islands and are relatively rare in other areas of the genome and the amplicons generated are of lower complexity, meaning that the hybridization signals will be stronger and more specific than in the case where enzymes with a higher frequency of restriction sites are used. Enzymes such as HpaII (C′CGG), AciI (C′CGC), HinP1I (G′CGC), and HpyCH4IV (A′CGT) may be especially convenient because they produce the same “sticky” ends, allowing subsequent linkage of the cleaved end of the DNA fragment to a single oligonucleotide-linker (i.e., a universal linker). A universal linker which can be ligated to all cleavage ends generated in a particular step of the method is preferred.

DNA fragments may be generated, at methylation sites that are non-methylated, by using a chemical agent other than a restriction enzyme. For example, DNA can be treated with an agent that specifically protects methylated DNA sites from cleavage (e.g., a methylation binding protein), and then the treated DNA is cleaved via an agent that cleaves the unmethylated methylation sites. Such an agent may be a methylation-insensitive restriction enzyme or a methyl-DNA binding protein that can be fused to or coupled with an endonuclease which can then specifically cut at the methylated CpG sites.

A “methylation insensitive agent” in this context is one that cleaves DNA at a cleavage site, regardless of whether the site is methylated or not. In a preferred embodiment, the methylation insensitive agent employed is a methylation-insensitive restriction enzyme. Exemplary of methylation-insensitive enzymes for use in the present invention are EcoRI (G′AATTC), ApoI (R′AATTY), Tsp509I (AATT), MseI (TTAA), BfaI (C′TAG), Csp6I (G′TAC), NIaIII (CATG′), DpnII (′GATC), CviJI (RG′CY), Sau3A (′GATC), RsaI (GT′AC), Tsp509I (′AATT), MaeI (C′TAG), NlAIII (CATG′), and DpnI (GA′TC). Other methylation insensitive agents may be chemicals, such as formic acid, hydrochloric acid, and performic acid. For example, an engineered agent such as a fusion protein comprising a methylation-specific DNA-binding protein and an endonuclease may be used for cleaving DNA at methylated and non-methylated methylation sites.

Ligating to Oligonucleotide-Linkers and Amplification

After generating DNA fragments by cleaving at non-methylated sites in the DNA, the ends of the fragments are ligated to an oligonucleotide-linker. An oligonucleotide-linker may be double- or single-stranded and generally is from about 12 to about 24 nucleotides in length, although shorter or longer oligonucleotide-linkers may be used. Double stranded oligonucleotide linkers are preferred. A double-stranded oligonucleotide-linker may be blunt-ended or have sticky ends. A blunt-ended oligonucleotide-linker may be linked to a blunt-ended DNA fragment. Preferable oligonucleotide-linkers of the invention have a sticky end that is complementary to a sticky end of a DNA fragment for which ligation is desired. Oligonucleotide-linkers may be prepared synthetically and, if double-stranded, may be designed with a sticky end by synthesizing top and bottom DNA stands of different length. In another approach, the linker may be synthetically prepared with a blunt end and then treated with an agent to create the appropriate sticky end. For example, a blunt ended oligonucleotide-linker may be treated with the same methylation sensitive restriction enzyme used to cleave the test or control DNA. In this case, a sticky end that is complementary will be generated on both the DNA fragment and the oligonucleotide-linker to be ligated to the fragment.

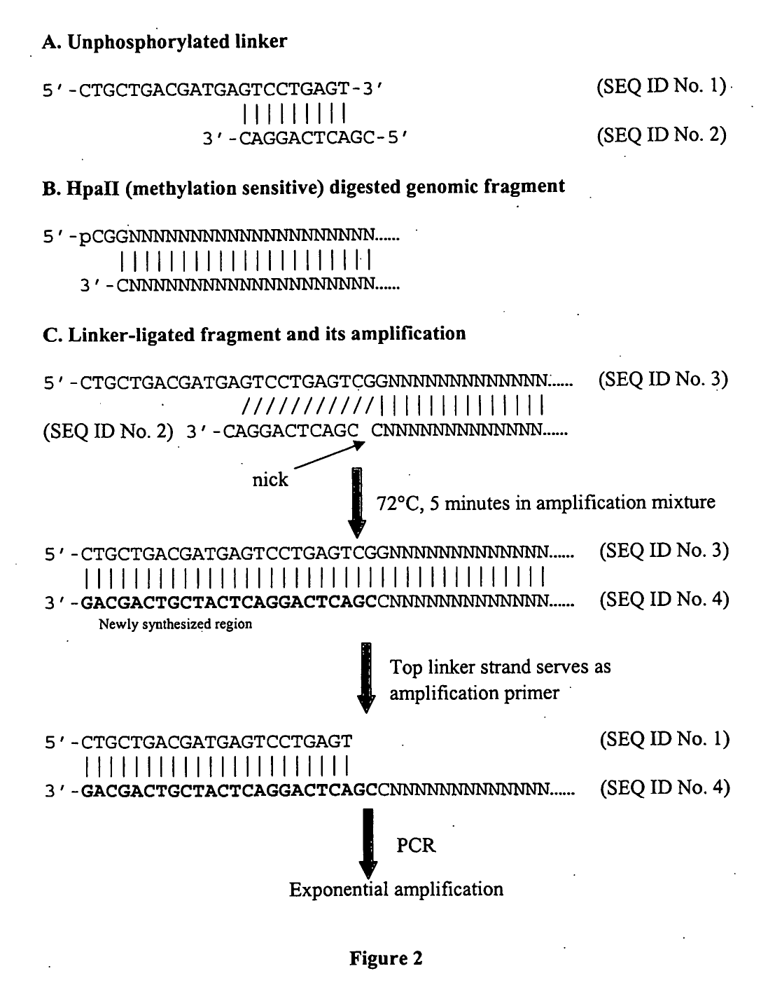

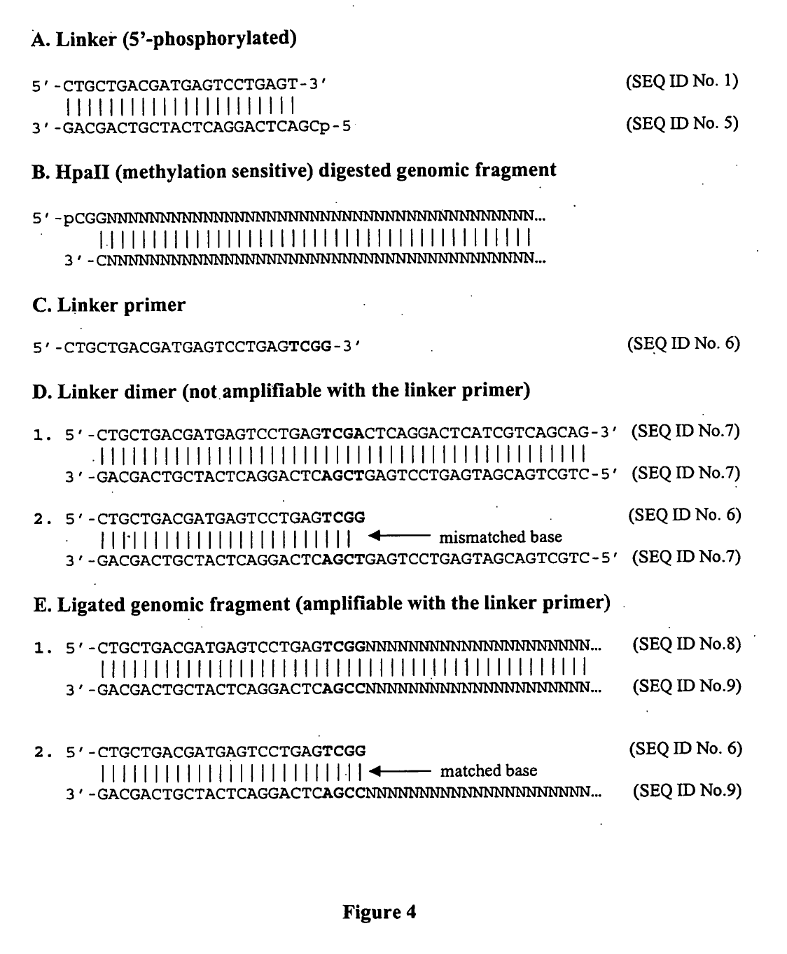

An exemplary double stranded oligonucleotide-linker for ligation to a HpaII-digested DNA fragment can be formed by the combination of SEQ ID NO:1 and SEQ ID NO: 2, shown below (the linker is shown at top of FIG. 2). This oligonucleotide linker is partially double stranded and not phosphorylated. An alternative oligonucleotide linker can be made by combining SEQ ID NO:1 with SEQ ID NO: 5 shown below (the linker is shown at top of FIG. 4). This oligonucleotide linker is fully double stranded except for a two base segment reflecting the HapII site. This second oligonucleotide linker also is phosphorylated at the 5′ end of the bottom strand as shown (see 5′ end of SEQ ID NO:5).

| | |
|---|---|
| 5′-CTGCTGACGATGAGTCCTGAGT-3′ | (SEQ ID NO:1; |
| | top strand) |
| 5′-CGACTCAGGA-3′ | (SEQ ID NO:2; |
| | bottom strand) |
| 5′-pCGACTCAGGACTCATCGTCAGCAG-3′ | (SEQ ID NO:5; |
| | bottom strand) |

With a phosphorylated double stranded oligonucleotide linker such as the combination of SEQ ID NO:1 and 5, both strands of the linker are ligated to the cut DNA (see FIG. 4, panel E1). With a non-phosphorylated double stranded oligonucleotide linker such as the combination of SEQ ID NO:1 and 2, only one strand of the linker is ligated to the cut DNA (e.g., the bottom strand as seen in FIG. 2, panel C). The other strand is held in place by Watson-Crick base pairing and may be removed by denaturing conditions.

The characteristics of the oligonucleotide-linker depend on the particular embodiment. For example, the oligonucleotide-linker may be labeled with a detectable moiety so that the DNA fragments may be detected in later steps of the method. Labeling may be accomplished, for example, by conjugating the detectable moiety directly to nucleotides in the linker or the fragment, or by designing the linker to have a unique site for labeling, or by post amplification random labeling. A unique site in the linker may be a chemical linker or a capture sequence of nucleotides. As used herein, a “capture sequence” of nucleotides is a sequence designed to anneal by base pairing with a target oligonucleotide sequence. For example, a capture sequence can be an oligonucleotide with an abiotic and GC-rich sequence that strongly base-pairs with a complementary sequence on another nucleic acid such as a dendrimer. An “abiotic” sequence is one which is not present in the genome of the test DNA. A dendrimer is a branched molecule to which dozens of dye molecules can be attached (Stears et al., Physiol Genomics 3: 93-99, 2000). A capture sequence can be designed into the oligonucleotide-linker to support labeling with a detectable moiety such as a labeled dendrimer, which is described in detail below. A post-amplification random labeling can be achieved using normal and fluorescent deoxynucleotides, a random primer, and a DNA polymerase as is well known in the art.

An oligonucleotide-linker also may be designed to provide a target for initiating synthesis of DNA during DNA amplification. Hence, after ligation to the linker, the ends of the DNA fragments can have a unique sequence which is of sufficient length to provide a unique recognition site for primers selected for in vitro DNA amplification, for example, by the polymerase chain reaction (PCR). Preferably, the oligonucleotide-linker contains a unique sequence sufficient for targeting by the amplification primer. The unique sequence may include a portion of the linker or may include all of the linker. An oligonucleotide linker may thus function as the linker primer. The amplification primer also may include some of the sequence of the adjoining DNA fragment.

The length of the amplification primers for use in the present invention depends on several factors including the nucleotide sequence and the temperature at which these nucleic acids are hybridized or used during in vitro nucleic acid amplification. The considerations necessary to determine a preferred length for an amplification primer are well known to the person of ordinary skill in the art. For example, the length of a short nucleic acid or oligonucleotide can relate to its hybridization specificity or selectivity. Because the digested fragments of DNA may contain a complex mixture of nucleic acids, primers which are shorter than about 12 nucleotides may hybridize to more than the linker, and accordingly would not have sufficient hybridization selectivity for amplifying only the DNA fragments cut with a methylation-sensitive agent. However, a 12- to 15-nucleotide sequence generally is represented only once in a mammalian genome. Sambrook et al. (1989), supra, at pages 11.7-11.8. Accordingly, to eliminate amplification of DNA fragments that are not ligated to oligonucleotide-linkers, amplification primers are chosen which are generally at least about 14 or 15 nucleotides long. Preferably, the primers are at least about 16-17 nucleotides long, and may even be 20-25 nucleotides long or longer. (Sambrook et al., pp. 11.7-11.8). An example of an amplification primer for a HaplI oligonucleotide linker is the following:

| | |
|---|---|
| 5′-CTGACGATGAGTCCTGAGTC-3′ | (SEQ ID NO. 6, |
| | linker primer) |

This primer is suitable for hybridizing and priming at the oligonucleotide-linker shown above (e.g., the double stranded linker formed using SEQ ID NOs: 1 and 5).

Ligation of oligonucleotide-linkers to DNA is accomplished using methods well known in the art. For example, see Sambrook et al. (1989), supra, at 1.53. This typically involves annealing of the oligonucleotide-linker to the DNA fragment, by gradual cooling from 50° C. to 25° C., followed by ligation using 400 U of T4 DNA ligase at 16° C. The step of gradual cooling can be eliminated if blunt-ended ligation is contemplated.

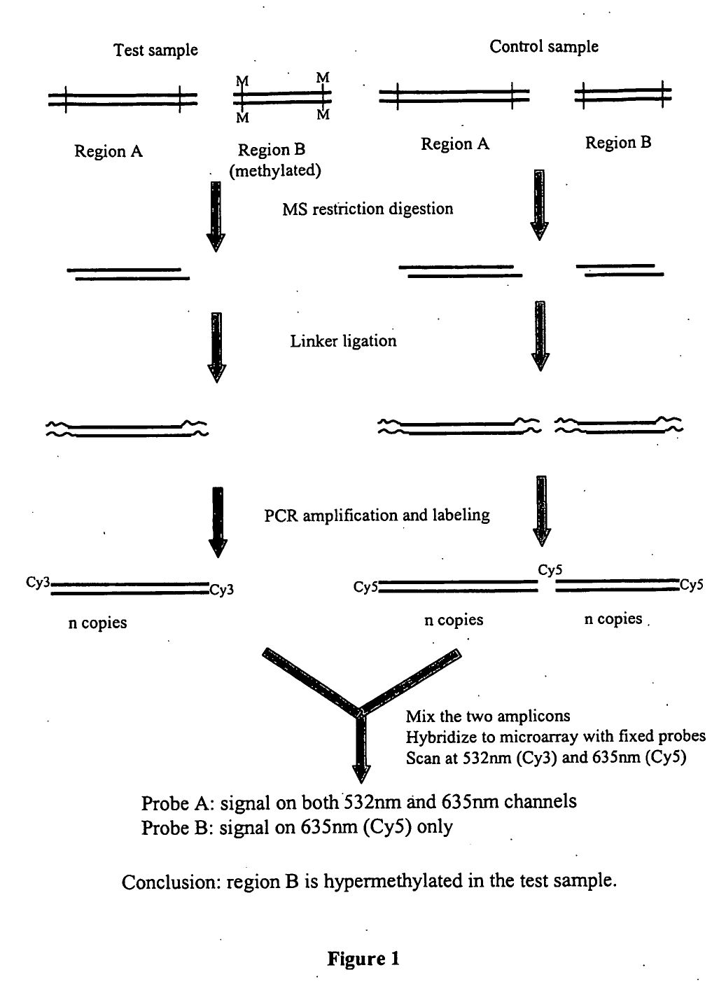

By ligating an oligonucleotide-linker to both ends of a DNA fragment generated by cleavage with a methylation sensitive agent, the ligated DNA fragment will be replicated from both ends when such ends are targeted by the amplification primer, resulting in efficient amplification, for example, by PCR (FIG. 1). Since only relatively short fragments (e.g., those less than 1000 base pairs) can be amplified under certain experimental conditions, the amplified fragments represent regions that are not methylated. Conversely, when a region of the DNA is methylated or has no CpG dinucleotides, it will not be cleaved by a methylation-sensitive agent, no oligonucleotide-linker will be ligated, and no amplification will result (e.g., FIG. 1). Therefore, the relative amount of amplified fragments from specific region of test and control samples will reflect their relative methylation level difference.

The polymerase chain reaction is a preferred method for DNA amplification. PCR synthesis of DNA fragments occurs by repeated cycles of heat denaturation of DNA fragments (i.e., heating to at least about 95° C.), incubation at a temperature permitting hybridization of the amplification primers to all or part of the primer adapter ends of the methylation-sensitive cut DNA fragment, and primer extension. These cycles can be performed manually or, preferably, automatically. Thermal cyclers such as the Perkin-Elmer Cetus® cycler (Perkin-Elmer Corp., Boston, Mass.) are specifically designed for automating the PCR process and are preferred. The number of cycles per round of synthesis generally varies from 2 to more than 50, the optimum number being readily determined by considering the source and amount of the nucleic acid template, the desired yield, and the procedure to be used for detection of the DNA. Heat stable amplification enzymes such as pwo, Thermus aquaticus or Thermococcus litoralis DNA polymerases are commercially available and eliminate the need to add enzyme after each denaturation cycle. Basic PCR techniques are described in U.S. patents by Saiki et al., see also 1988 Science 239: 487, and by U.S. Pat. No. 4,683,195, No. 4,683,202 and No. 4,800,159, and other PCR variations well-known to the field.

The precise conditions for PCR and hybridization have a much reduced effect on the outcome of present invention as compared to that in other PCR-based assays in cases where both amplification and hybridization reactions are competitive: the DNA from the test and control sample is combined and amplified simultaneously, thus, competing equally for amplification resources like the polymerase and nucleotide triphosphates. Therefore, more accurate assays are attainable with the present invention, and more variation or error in performing the assay is tolerated without adversely affecting the results. Nevertheless, the variables generally affecting PCR including temperature, salt and cation concentration, pH, and related conditions may be optimized for each situation.

A variety of enzymes suitable for DNA amplification such as in PCR are well known in the art and include, for example, Pwo, Escherichia coli DNA polymerase I, Klenow fragment of E. coli DNA polymerase I, T4 DNA polymerase, T7 DNA polymerase, Thermus aquaticus (Taq) DNA polymerase, Thermococcus litoralis DNA polymerase, SP6 RNA polymerase, T7 RNA polymerase, T3 RNA polymerase, T4 polynucleotide kinase, Avian Myeloblastosis Virus reverse transcriptase, Moloney Murine Leukemia Virus reverse transcriptase, T4 DNA ligase, E. coli DNA ligase or Qβreplicase, and the like. Mixtures of enzymes also can be used (e.g., Pwo and Taq). Preferred amplification enzymes are the Pwo and Taq polymerases because of their high fidelity and high processibility. Other DNA-amplification enzymes, known and yet to be discovered, may be utilized in the present invention.

It is desirable to use conditions for amplification such that the DNA fragments differing in GC content can be amplified to a similar extent. Addition of betaine (1.2 M) to a PCR reaction can be used to assist in the amplification of GC-rich regions. Under such conditions, a region of 42% GC and region of 73% GC can be amplified about equally well. Other approaches to achieve balanced amplification of GC rich and GC poor fragments are known in the art.

In situations where the site to be analyzed can be cleaved as a fragment (suitable for amplification) with a methylation sensitive agent, the oligonucleotide linker can be phosphorylated or unphosphorylated. A scheme for use of a phosphorylated oligonucleotide linker is shown in FIG. 1. When a phosphorylated oligonucleotide-linker with sticky ends is used, the possibility of forming dimers of the oligonucleotide-linkers can occur and such dimers can be ligated during the ligation step. Linker dimers may hybridize to amplification primers and be amplified, thus depleting the resources of the amplification reaction, thereby reducing the amount of amplification of ligated DNA fragments. Different strategies for minimizing this problem are available and include, for example: (1) Using column chromatography, such as with a silica-gel-membrane type spin column, to purify the ligated DNA fragments from the linker dimers. In this approach, further elimination of linker dimers may be achieved by washing the column with 30% guanidine hydrochloride prior to the regular washing step; and (2) Using a linker primer that is comprised of a 3′-mismatched nucleotide when hybridized to the linker dimer but a 3′ matched nucleotide when hybridized to the linker-ligated genomic fragment. An example of the second approach is shown in FIG. 4.

A scheme for amplification when a unphosphorylated oligonucleotide linker is used is shown in FIG. 2. In this embodiment, the linker is designed as shown with a short lower strand that is not phosphorylated. Thus, following the step of ligation, a nick or space will be present between the lower strand of the linker and the fragment. Primer amplification may be achieved following ligation by a short chain extension reaction step in which polymerase and NTPs are added to the linked fragment and the mixture incubated (e.g., 72° C.) for several minutes. During the polymerization step, the unligated bottom strand is denatured and removed allowing the polymerase to synthesize a bottom strand flush to the end of the top strand. Exponential amplification may be achieved by application of cycling temperatures. In this case, there is no need to add a linker primer since excess top strand from the oligonucleotide linker that remains from the ligation step can serve as the PCR primer.

In the case when a particular methylation site to be interrogated is located too far from the next nearest methylation site, making it difficult to amplify and thus detect a fragment of such length, the DNA can be cleaved with both a methylation sensitive and a methylation insensitive agent to generate amplifiable fragments. The methylation sensitive and insensitive cleavage agents may be used separately or at the same time if compatible. A particular methylation insensitive agent can be chosen (preferably a methylation-insensitive restriction enzyme) to provide a second cleavage site nearer to the site cleaved by the methylation-sensitive agent, yielding a fragment size more suited to amplification. Generally, fragments lengths of 50-1000 base pairs will be suitable for amplification.

A preferred methylation insensitive agent is a methylation insensitive restriction enzyme. The methylation insensitive restriction enzyme preferably is chosen to produce a different sticky end on the DNA than the end generated by the methylation-sensitive agent. In this case, oligonucleotide-linkers that are distinct from each other and which ligate only to one of the types of sticky ends generated should be used.

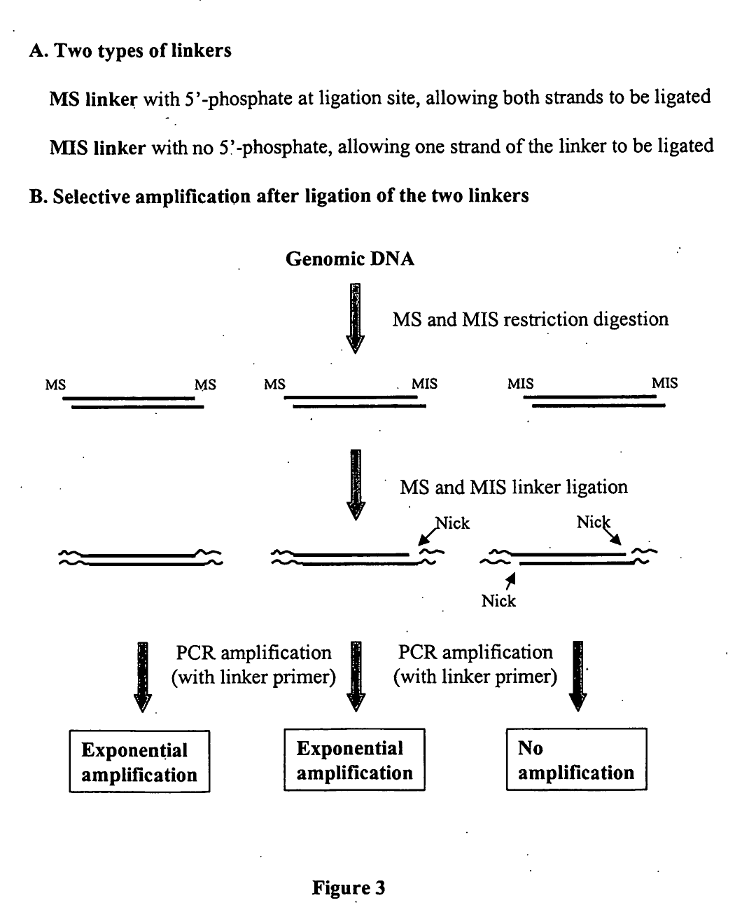

To avoid amplifying fragments generated solely by cleavage with the methylation insensitive agent, one may use a phosphorylated oligonucleotide linker for ligation to the methylation sensitive site and a unphosphorylated oligonucleotide linker for ligation to the methylation insensitive site. This approach is illustrated further in FIG. 3. As is seen in the figure, following ligation to the methylation insensitive site, a gap or “nick” remains between one strand of the linker and the fragment. The gap prevents amplification of the strand by a linker primer chosen to hybridize to that strand. Under such circumstance, exponential amplification will occur for DNA fragments resulting from cleavage at both ends by a methylation-sensitive agent and for DNA fragments resulting from cleavage at one end by a methylation-sensitive agent and at the other end by a methylation-insensitive agent. DNA fragments resulting from cleavage at both ends by a methylation insensitive enzyme will not be amplified.

When both a methylation-sensitive agent and a methylation-insensitive agent are used to cleave the DNA, oligonucleotide-linkers used preferably will selectively ligate only to the ends generated by one or the other agent. Selective ligation may not be needed when each of the DNA cleavage agents are used in a separate reaction and the oligonucleotide-linker for the first reaction is added before the DNA is exposed to the second cleavage agent.

In an alternative embodiment, the step of DNA amplification may be dispensed with entirely. In this case, the ends of the DNA fragments generated by cleavage with the methylation sensitive agent are ligated to an oligonucleotide-linker, the DNA fragments are size selected to isolate a particular size range, the fragments are labeled with a detectable moiety, and then hybridized and analyzed as described further ahead. The DNA fragments are size selected, preferably to a size range of about 2,500 base pairs or less in length, more preferably about 1,500 base pairs in length or less and even more preferably about 1,000 base pairs in length or less. DNA fragments may be size separated before or after ligation to oligonucleotide-linkers. Sizing may be achieved by any method known in the art, including, gel electrophoresis, column chromatography, density gradient centrifugation or ultracentrifugation (see, e.g. Steensel et al., (2001) Nature Genetics 27:304-308), filtration through membranes of controlled pore size, and the like. Filtration through membranes of controlled pore size may be achieved by applying an external pressure or by centrifugation.

In the embodiment without amplification, the detectable moiety is preferably attached to the oligonucleotide-linker and the moiety is preferably of suitably high specific activity. A labeled dendrimer or a fluorescent resonant energy transfer dye are examples of high specific activity detectable moieties. The oligonucleotide linker can have a capture sequence designed for binding to a detectable moiety such as a labeled dendrimer. The oligonucleotide-linker may be labeled before or after it is ligated to the DNA fragments.

The amount of DNA digested may be increased to improve detection sensitivity in embodiments that do not use amplification. DNA in the range of 0.5-1.5 mg may be suitable for this purpose, although persons of ordinary skill in the art realize that the quantity of DNA sufficient to allow omitting the DNA amplification step depends on other features of the assay and can be readily determined without resort to undue experimentation.

In yet another embodiment, the step of DNA amplification and the step of ligating the DNA fragments to an oligonucleotide-linker may be dispensed by using a sufficiently large amount of the DNA, size-fractionating the digested fragments, optionally labeling the fragments through random priming (e.g., with Cy3 and Cy5 dye-labeled dCTP), and hybridizing them to a solid support such as a microarray. For example, see Steensel et al., Nature Genet. 27: 304 (2001).

Labeling DNA

DNA can be labeled with a detectable moiety to improve detection in later steps. Labeling can occur at any of a variety of steps in the method. For example, the detectable moiety may be attached to the oligonucleotide-linker, which is then ligated to the DNA fragment. In another approach, the detectable moiety may be attached to the amplification primer, which is incorporated into the amplified DNA. In yet another approach, the DNA fragment may be directly labeled with the detectable moiety. In some situations, it may be preferable to label with a detectable moiety at more than one step (e.g., use a labeled oligonucleotide-linker and direct labeling). This may be useful when increased sensitivity is desired. Various combinations of detectable moieties also may be used to label the same molecule to increase its specific activity.

The phrase “detectable moiety” is used here to denote any molecule (or combinations of molecules) that may be attached or otherwise associated with a molecule so that the molecule can be detected indirectly by detecting the detectable moiety. A detectable moiety can be a radioisotope (e.g., iodine, indium, sulfur, hydrogen etc.) a dye or fluorophor (e.g., cyanine, fluorescein, rhodamine), protein (e.g., avidin, antibody), enzyme (peroxidase, phosphatase, etc.), or any other agent that can be detected directly or indirectly. An enzyme is an example of a detectable moiety detected by indirect means. In this case, the enzyme is attached to the target nucleic acid and the presence of the enzyme is detected by adding an appropriate substrate that when acted upon by the enzyme, causes the substrate to change in color or to release a cleavage product that provides a different color from the original substrate.

A fluorescent detectable moiety can be stimulated by a laser with the emitted light captured by a detector. The detector can be a charge-coupled device (CCD) or a confocal microscope, which records its intensity. In the case of an array, the intensities provided by the array image can be quantified by measuring the average or integrated intensities of the spots. Interpreting the data from a microarray experiment can be assisted using special software, such as Dapple; see (http://www.cs.washington.edu/homes/jbuhler/research/dapple/).

A preferred detectable moiety is a cyanine dye such as Cy-5 and Cy-3. For example, test DNA fragments can be labeled with Cy-5, while control DNA fragments can be labeled with Cy-3 (or vice versa). In this way, the two DNA fragment preparations may be mixed and then hybridized together to the solid support, scanned with the above devices at the corresponding wavelength for each dye, with the measured densities providing the ability to distinguish the relative amounts of each DNA fragment that hybridized from the two samples.

A detectable moiety may include more than one chemical entity such as in fluorescent resonance energy transfer (FRET). In FRET based assays, interaction between biomolecules is measured indirectly by conjugating one of a pair of carefully selected fluorescent dyes to each of the molecules of interest. The absorption spectrum of the acceptor must overlap fluorescence emission spectrum of the donor, and donor and acceptor transition dipole orientations must be approximately parallel. In FRET, when these fluorescent dyes are held in close proximity (typically 10-100 Å) due to binding of the biomolecules, a unique fluorescence signal is developed that specifically confirms the proximity and thus the binding reaction. Resonance transfer results an overall enhancement of the emission intensity. For instance, see Ju et. al. (1995) Proc. Nat'l Acad. Sci. (USA) 92: 4347. To achieve resonance energy transfer, the first fluorescent molecule (the “donor” fluor) absorbs light and transfers it through the resonance of excited electrons to the second fluorescent molecule (the “acceptor” fluor).

In most cases, when donor and acceptor dyes are different, FRET can be detected by the appearance of sensitized fluorescence of the acceptor or by quenching of donor fluorescence. When the donor and acceptor are the same, FRET can be detected by the resulting fluorescence. Donor/acceptor pairs of dyes that can be used include, for example, fluorescein/tetramethylrohdamine, IAEDANS/fluroescein, EDANS/DABCYL, fluorescein/fluorescein, BODIPY FL/BODIPY FL, and Fluorescein/ QSY 7 dye. See, e.g., U.S. Pat. No. 5,945,526 to Lee et al. Many of these dyes also are commercially available, for instance, from Molecular Probes Inc. (Eugene, Oreg.).

In another embodiment, signal amplification may be achieved using labeled dendrimers as the detectable moiety. A dendrimer is a bulky three dimensional molecule that can be labeled to very high specific activity, providing signal amplification (see, e.g., Physiol Genomics 3:93-99, 2000). Fluorescently labeled dendrimers are available from Genisphere (Montvale, N.J.). These may be chemically conjugated to the DNA fragments by methods known in the art. The labeled dendrimer also can be attached to the amplified DNA by designing a capture sequence in the amplification primer (e.g., a long abionic, GC-rich nucleotide sequence), which can be targeted by a complementary sequence in the dendrimer. In the event the capture sequence in the primer creates problems during amplification, a polymerization “stop signal” can be added 3′ of the capture sequence (between the capture sequence and the primer sequence) so that the capture sequence in the primer will not be amplified. The C18 spacer below is an example of a useful “stop signal.” 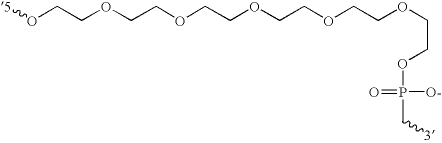
Persons of ordinary skill in the art will realize that this is just one example, and many other stop signals are available and known in the art. For example several may be obtained from Integrated DNA Technologies, Inc. (Coralville, Iowa) and other sources.
Hybridization to Immobilized Polynucleotides

A solid support with polynucleotides immobilized thereon is useful for capturing DNA fragments prepared as described above to determine methylation status for any region of the genome. The fragments are preferably labeled with a detectable moiety as already discussed to assist in detecting the amount of the DNA that hybridizes to the immobilized polynucleotides.

The immobilized polynucleotides are designed to specifically hybridize to a region of the DNA for which determination of its extent of methylation is desired. Thus, if one is interested in determining the extent of methylation at a single site in the genome, the solid support may have a polynucleotide complementary to a fragment that would arise in the method if that site were not methylated. A solid support may have multiple different immobilized polynucleotides, each designed to anneal to a different DNA fragment that may be generated in the assay depending on whether the methylation site at one end of the fragment is methylated. As described further below, multiple polynucleotides may be immobilized to a single support in the form of an array. The solid phase may have from hundreds to tens of thousands of polynucleotides immobilized as an array, depending on the size of the array and the technology used for immobilization.

Quantitation of the extent of methylation of one or more regions of DNA in a test sample versus a control sample is achieved through competitive amplification of fragments of DNA with single pairs of oligonucleotide-linkers (or one oligonucleotide-linker in the case of single enzyme digestion) and/or competitive hybridization to the solid support. This may be appreciated from the following example where genomic DNA from the test and control are digested with at least one methylation sensitive enzyme. If 100,000 fragments are amplified using oligonucleotide-linkers, and 100 of the fragments are differentially methylated between the two samples, 99,900 fragments will be amplified to very similar extent in both samples after the reactions are saturated, and the 100 fragments will be amplified differentially. After mixing the two amplified products (each labeled with a distinct dye), hybridizing to polynucleotides immobilized on the support (e.g., a microarray), and scanning at the specified wavelength, 99,900 wells or spots on the support will show neutral color (i.e., similar levels of hybridization signal to the two DNA sources) while 100 wells or spots will show a red (Cy-5) or green (Cy-3) color (i.e., differential levels of hybridization signal to the two DNA sources), the latter thereby indicating a difference in methylation level at those sites (e.g., CpG sites).

Using the combination of Cy3 labeled test sample and Cy5 labeled control sample, the resulting hybridization signal strength on the array following hybridization can be interpreted as follows:
Red: Means differential methylation between the test sample and control sample. The control sample dominates, so the control sample is less methylated than the test sample.
Green: Means differential methylation between the test sample and the control sample. The test sample dominates, so the test sample is less methylated than the control sample.
Yellow: Means about equal methylation for the test sample and the control sample.
No color: Means either heavy methylation in both test and control samples or the immobilized polynucleotide sequence is not present in the genomic DNA of the test or control.
Color intensity: Although less predictive than color, a high intensity indicates a low overall extent of methylation for the region in both the test and control, while a low intensity indicates a high overall extent of methylation for the region in both the test and control.

A. Immobilized Polynucleotides

The sequence of polynucleotides immobilized to a support can be determined by resort to DNA sequence readily available in publicly available sequence databases, including that gleaned from the Human Genome Project. As used herein, a polynucleotide can be a single or a double stranded nucleic acid and may be DNA or RNA. A polynucleotide includes at least 2 nucleotides and may be prepared synthetically or by cleaving natural nucleic acid. The term polynucleotide encompasses the term oligonucleotide, which refers to a shorter polynucleotide of between two to about 100 nucleotides in length.

Polynucleotide sequences can be designed to hybridize to virtually any region of the genome where methylation is suspected. A virtual (i.e., “computer based”) restriction digestion may be performed on human genomic DNA sequence using the recognition sequence for the methylation-sensitive agents of interest, and the identity of sequence for the immobilized polynucleotides determined. Immobilized polynucleotides can be selected to interrogate particular sequences in genes, such as the upstream regulatory region of a gene, the protein encoding portion of the gene, or downstream sequence associated with the gene. In such cases, the immobilized polynucleotides may be designed to hybridize to a sequence in the gene or near to the gene.

The immobilized polynucleotide may be a short segment of DNA such as an oligonucleotide or the immobilized polynucleotide can be a longer stretch of DNA such as a cDNA or portion thereof or genomic fragment of DNA. Polynucleotides also may be chosen to interrogate intergenic regions, which are referred to herein as sequence upstream of a promoter and downstream of a poly A site. In either case, the sequence of immobilized polynucleotides is preferably selected to hybridize to the region of interest, but not to other regions. This can be determined with the aid of computer sequence programs such as BLAST.

Polynucleotides for immobilization should be designed such that they will not hybridize to other fragments that may be generated by the particular embodiment of the method chosen. In the case where amplification is used, the immobilized polynucleotides preferably have sequence complementary to a segment of DNA that have potential cleavage sites for a methylation-sensitive agent. The sites should be close enough to each other so that the fragment can be amplified under the specified PCR conditions if the sites are not methylated, and, therefore, cleaved. Polynucleotides for immobilization are preferably oligonucleotides which comprise a hybridizing sequence preferably between about 40 to 70 nucleotides in length and more preferably between about 50 to 60 nucleotides in length. As already discussed, immobilized polynucleotides can be longer than 100 nucleotides in length and may even constitute a cDNA or genomic DNA fragment thereof.

B. Solid Support

Polynucleotides immobilized to a solid support are used for detecting hybridization to the amplified and detectably-labeled target nucleic acid. The polynucleotides may be immobilized on any solid support, organic or inorganic, or a combination of any of these—in the form of particles, strands, precipitates, gels, sheets, tubing, spheres, containers, capillaries, pads, slices, films, plates, slides, and the like. Typical supports are made of glass, plastic, or nylon.

With respect to implementing the present invention, a solid support for immobilizing polynucleotides preferably is flat but may take on alternative surface configurations. For example, the solid support may contain raised or depressed regions on which polynucleotide synthesis takes place or where polynucleotides are attached. In some embodiments, the solid support will be chosen to provide appropriate light-absorbing characteristics. Thus, the support may be a polymerized Langmuir Blodgett film, functionalized glass, Si, Ge, GaAs, GaP, SiO₂, SiN₄, modified silicon, or any one of a variety of gels or polymers such as (poly)tetrafluoroethylene, (poly)vinylidendifluoride, polystyrene, polycarbonate, or combinations thereof. The solid support may be a glass microscope slide.

The surface of the solid support can contain reactive groups, which could be carboxyl, amino, hydroxyl, thiol, or the like suitable for conjugating to a reactive group associated with the immobilized polynucleotides. Polynucleotides can be attached to the solid support by chemical or physical means such as through ionic, covalent or other forces well known in the art. Polynucleotides also can be attached to a solid support by means of a spacer molecule, essentially as described in U.S. Pat. No. 5,556,752 to Lockhart el al. A spacer molecule typically comprises between 6-50 atoms in length although larger and shorter spacers are possible and includes a surface attaching portion that attaches to the solid support.

Attachment to the support can be accomplished by carbon-carbon bonds, using supports having, for instance, (poly)trifluorochloroethylene surfaces or, preferably, by siloxane bonds, employing glass or silicon oxide as the solid support, for example. Siloxane bonding can be formed by reacting the support with trichlorosilyl or trialkoxysilyl groups of the spacer. Aminoalkylsilanes and hydroxyalkylsilanes, bis(2-hydroxyethyl)-aminopropyltriethoxysilane, 2-hydroxyethylaminopropyltriethoxysilane, aminopropyltriethoxysilane or hydroxypropyltriethoxysilane are useful surface attaching groups. Additionally, for use in synthesis of polynucleotides, the spacer can have a protecting group, attached to a functional group (i.e., hydroxyl, amino or carboxylic acid) on the distal or terminal end of the spacer (opposite the solid support). After deprotection and coupling, the distal end can be covalently bound to an oligomer.

Another solid support is a microelectronic chip as described, for example, in U.S. Pat. No. 6,051,380 to Sosnowski et al., U.S. Pat. No. 6,129,828 to Sheldon, III et al., and U.S. Pat. No. 6,017,696 to Heller. This type of chip exploits electronically accelerated hybridization conducted under very low salt conditions, which avoids problems, with DNA conformation and secondary structure, associated with other hybridization methods. Immobilized polynucleotides can be moved electronically to specific sites on the microchip, and then hybridization is determined. In the electronic mediated approach, use of an electronically mediated, active hybridization process to move and concentrate target DNA molecules reduces the time to detect hybridization from hours (as in conventional, passive methodology) to minutes.

The polynucleotides can be attached, via conventional technology, to a solid support in the form of a microarray, also known as a “DNA chip,” a “DNA microarray,” a “gene array”, a “gene chip,” and a “genome chip,” or in the form of a macroarray. In this context, an array is an orderly arrangement of samples that enables the matching of known and unknown DNA samples, founded on base-pairing rules, and the automation of identifying the unknowns.

“Microarray” and “macroarray” are relative terms, distinguished from each other in terms of the diameter of the sample spots involved. Thus, microarray sample spots typically are about 200 microns in diameter or less, making it possible to array thousands of sample spots on a single chip. These arrays can be prepared by hand but, preferably, are made using specialized robotics and read by means of specialized imaging equipment, particularly with spots with diameters in the lower-end range. By contrast, macroarray sample spots typically are greater than 200 microns in diameter, making them suitable for imaging by gel and blot scanners. Macroarrays may be prepared by hand using standard microplates or standard blotting membranes.

A microarray containing immobilized polynucleotides can be prepared by a number of well-known approaches including, for example, light-directed methods, such as VLSIPS™ (see U.S. Pat. No. 5,143,854), mechanical methods such as described in PCT No. 92/10183 or U.S. Pat. No. 5,384,261, bead-based methods described, for example, in PCT US/93/04145, and pin-based methods as detailed in U.S. Pat. No. 5,288,514, inter alia. U.S. Pat. No. 5,556,752 to Lockhart describes the preparation of a library of different, double-stranded polynucleotides as a microarray, using the VLSIPS,™ and this approach also is suitable for preparing a library of polynucleotides in a microarray. Flow channel techniques, as described U.S. Pat. No. 5,677,195 and No. 5,384,261, also can be employed to prepare a microarray chip that has a variety of different immobilized polynucleotides. In this case, certain activated regions of the substrate are mechanically separated from other regions when the polynucleotides are delivered through a flow channel to the support. As noted, the Lockhart '752 patent describes flow channel methodology in some detail, including the use of protective coating-wetting facilitators to enhance the directed channeling of liquids though designated flow paths.

Spotting methods also can be used to prepare a microarray biochip with a variety of immobilized polynucleotides. In this case, reactants are delivered by directly depositing relatively small quantities in selected regions of the support. In some steps, of course, the entire support surface can be sprayed or otherwise coated with a particular solution. In particular formats, a dispenser moves from region to region, depositing only as much polynucleotides or other reagent as necessary at each stop. Typical dispensers include a micropipette, nanopippette, ink-jet type cartridge or pin to deliver the polynucleotide containing solution or other fluid to the support and, optionally, a robotic system to control the position of these delivery devices with respect to the support. In other formats, the dispenser includes a series of tubes or multiple well trays, a manifold, and an array of delivery devices so that various reagents can be delivered to the reaction regions simultaneously.

Spotting methods are well known and include, for example, those described in U.S. Pat. No. 5,288,514, No. 5,312,233 and No. 6,024,138. In some cases, a combination of flowing channel and “spotting” on predefined regions of the support also can be used to prepare microarray biochips with immobilized polynucleotides.

Information and examples of constructing microarrays are readily available in the art, such as in Cheung et al., Nature Genetics, Vol. 21 (suppl.), pp. 15-19 (1999); Lipshutz et al., Nature Genetics, Vol. 21 (suppl.), pp. 20-24 (1999); Brown & Botstein, Nature Genetics, Vol. 21 (suppl.), pp. 33-37 (1999); Shoemaker et al., Nature, Vol. 409, pp. 922-27 (2001); Schena et al., Science (1995) 467-470; Schena et al., P.N.A.S. U.S.A. (1996) 93: 10614-10616; Pietu et al., Genome Res. (June 1996) 6: 492-503; Zhao et al., Gene (Apr. 24, 1995) 156: 207-213; Soares, Curr. Opin. Biotechnol. (October 1997) 8: 542-546; Raval, J. Pharmacol Toxicol Methods (November 1994) 32: 125-127; Chalifour et al., Anal. Biochem (Feb. 1, 1994) 216: 299-304; Stolz & Tuan, Mol. Biotechnol. (December 1996) 6: 225-230; Hong et al., Bioscience Reports (1982) 2: 907; and McGraw, Anal. Biochem. (1984) 143: 298.. Numerous patents related to microarray development include U.S. Pat. No. 5,082,830, No. 6,110,426, No. 6,004,755, No. 5,445,934, No. 5,532,128, No. 5,556,752, No. 5,242,974, No. 5,384,261, No. 5,405,783, No. 5,412,087, No. 5,424,186, No. 5,429,807, No. 5,436,327, No. 5,472,672, No. 5,527,681, No. 5,529,756, No. 5,545,531, No. 5,554,501, No. 5,561,071, No. 5,571,639, No. 5,593,839, No. 5,599,695, No. 5,624,711, No. 5,658,734 and No. 5,700,637, and PCT application WO 97/27317.

C. Hybridization and Analysis

Hybridization conditions (either initial hybridization without washing or a combination of initial hybridization and washing) can be selected that allow the DNA fragments to hybridize the complementary immobilized polynucleotides of the solid support and avoid unwanted cross hybridization. See e.g., Sambrook (1989), Chapter 9.47-9.62. In general, DNA annealing is carried out in high ionic strength to maximize the annealing rate and at temperatures about 20-25° C. below the melting temperature of the DNA (Tₘ). Various agents may be used to reduce non-specific binding (e.g. Denhardt's solution, BLOTTO, heparin, and denatured salmon sperm DNA). Washing conditions should be as stringent as possible, generally sufficient salt and at a temperature 12-20° C. below the Tₘ. Washing conditions may be determined empirically in preliminary experiments. Other approaches may be used and may readily be determined by one of ordinary skill in the art.

The amount of amplified DNA that hybridizes to the solid support is compared to a control to determine whether the extent of methylation of one or more regions of DNA in a test sample is different from the control sample. In one embodiment, the control can be an actual sample of DNA such as genomic DNA from a normal healthy tissue that is processed in parallel with the test sample. Such control sample may be obtained from the same type of cell, tissue or organ as that from which the test sample was obtained. Alternatively, a control sample may contain one or more selected DNA fragments, which may be prepared so that it is fully methylated, fully unmethylated, or partially methylated as desired. The control also can be a theoretical control which represents a historical value derived from prior experimentation.

In a preferred embodiment, test and control DNA fragments are separately labeled with Cy-3 or Cy-5 dye. The labeled test and control fragments are then mixed together and hybridized to the solid support. After washing, the slide is scanned with an array scanner either successively or simultaneously at the excitation wavelength of 635 nm (Cy5) and 532 nm (Cy3). The images for each wavelength are recorded, pseudo-colors are applied to each image (red for Cy5 and green for Cy3) and the images are overlaid to show the relative density differences. The densities at both wavelength for each spot are also quantified and analyzed. The extent of methylation (“methylation status”) of DNA in a test sample relative to a control sample is then determined either visually by the color of each spot or through the measured density for each scanned channel.

Due to the qualitative nature of the present method achieved through the competitive amplification and hybridization, a very high degree of sensitivity, enabling the detection of subtle changes in methylation level for each region is attainable. This is highly desirable because tissue samples often contain mixed cell populations.

Applications of the Method

The present invention provides a high throughput methodology for detecting methylation changes at specific regions throughout the genome. High throughput can be accomplished using well known systems involving robotic control, automated liquid handling, automatic spotting, plate and slide loaders, pin arrays (96, 384, 1536 etc.), and the like (see e.g., automated array systems from Cartesian Technologies Inc.). This will allow tens of thousands of such regions, both CpG islands and non-island regions, to be tested simultaneously so that any methylation change in the genome can be detected. Also, hundreds or more samples from different individuals can be analyzed, and changes in methylation that are linked with a particular disease can later be exploited as a diagnostic screen for the disease. Other applications for the present invention are detailed below.

Diagnosis: The information thereby gleaned from using the methods herein informs diagnoses and prognoses that reflects, in whole or in part, the gene-silencing pattern characterizing a particular disease. That is, the methylation determined via the present invention offers a disease-state indicator of exceptional selectivity, specificity, and sensitivity. Furthermore, aberrant CpG island-hypermethylation, which occurs at high frequency in tumors, can yield diagnostic information as well. Determining patient's genome methylation by means of the present invention opens the way, in a cost-effective manner, for an unprecedented early warning diagnosis. Inappropriate methylation changes in CpG islands is one of the earliest known markers in the development of many cancers and direct detection of these molecular aberrations using the methods disclosed herein provides an extraordinary opportunity for unprecedented early stage molecular diagnosis of cancer. In addition, epigenetic changes have been implicated in several other important diseases. These include atherosclerosis (Post et al., 1999), Angelman syndrome (Lalande et al., 1999), Duchenne muscular dystrophy (Yoshioka et al., 1998) and ICF syndrome (Kondo et al., 2000), to name a few.

Enabling technology for improved clinical trials: The methods disclosed herein can be used to determine which individuals are afflicted with methylation dependent cancers. This can increase the success of efficacy studies in clinical trials of drugs targeting the basis for the methylation difference in the cancer. Drug candidates that effect methylation status represent the next generation of non-cytotoxic cancer therapies. In addition, since methylation status of a gene often correlates with its expression, methylation profile of test subjects can also be used in patient selection in clinical trials of other drugs, including cancer drugs.

Personalized Medicine: With the high cost of cancer therapies and the wide variety of cancer types, it is important that tests be developed to determine which patients will respond to which therapies. As the new generation of methylation dependent cancer therapies advances, the assays of the present invention will be important to determine which cancer patients are afflicted with methylation defects. Such data can help the oncologist's therapy decision process, determining patient suitability for a methylation-based drug regimen.

Discovery: Detection of inappropriate methylation in CpG islands acts as an indicator of which genes are involved in the development of cancer. Furthermore, only a small fraction (<3%) of all CpG islands have been investigated as to their role in cancer. Discoveries of new gene silencing events in cancer using the methods described herein will provide critical information for the initiation of new drug discovery and optimization strategies.

Toxicology: The cost of bringing drug candidates through clinical trials that eventually fail due to toxicological problems is enormous. Thus, there is a great need for methylation detection methods to “weed-out” drugs with toxicology problems at an early (pre-clinical) stage. Applying the methods of the present invention in a high throughput screening format will be helpful in determining if a particular drug impacts the methylation status of cells or tissues. Such screening would lower the likelihood that candidate drugs with mutagentic or epigenetic-based toxicity will proceed inappropriately to clinical trials.

### EXAMPLES

### Example 1

### Direct Microarray Screening of Differentially Methylated Sites (“DMS²) for a Mouse Transgene

This example demonstrates the use of an embodiment of the present invention for detecting the extent of methylation of a transgene in mouse DNA. The method involves a DNA amplification step and is referred to as DMS² (direct microarray-based screening of differentially methylated sites).

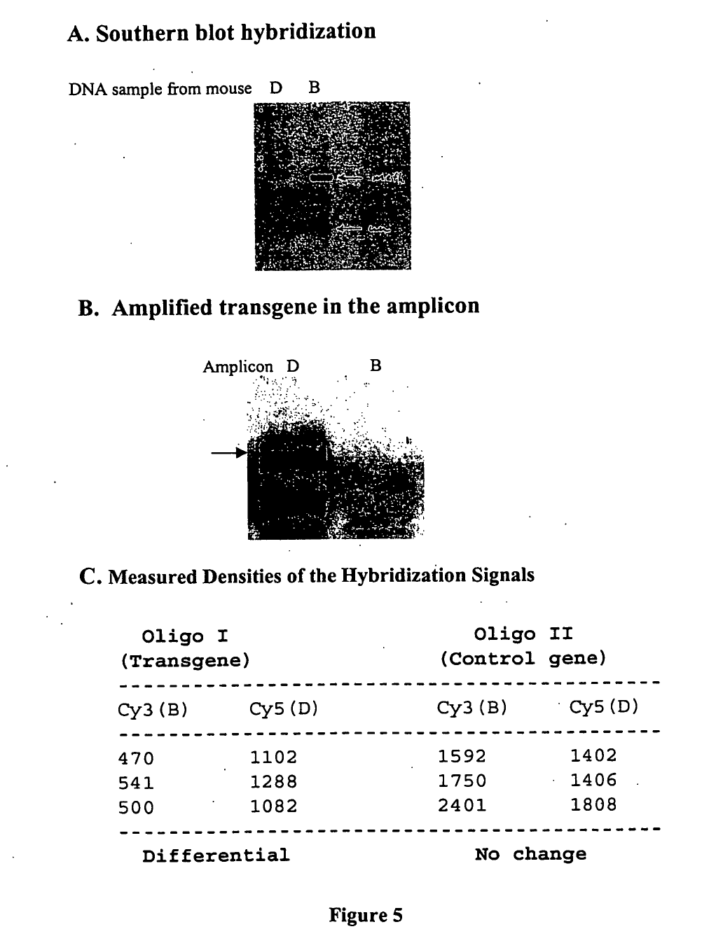

Oligonucleotides of 50-60 nucleotides in length were synthesized for hybridizing to selected CpG islands in mouse genes and for hybridizing to a mouse transgene known as HRD (Engler et al., (1991) Cell 65(6):939-47). HRD provides a useful control DNA in the assay because the transgene is highly methylated in one mouse strain (C57BL/6J, abbreviated as B) and unmethylated in another (DBA/2J, abbreviated as D), and HRD is integrated in the mouse genome in amounts comparable with other mouse genes. FIG. 5A is a Southern blot that demonstrates that the transgene is methylated in strain B but not in strain D.

DNA (1 μg) from each HRD transgenic mouse strain was separately digested with five units of HpaII in 50 μl for 6 hours at 37° C. The digestion mixture was heat inactivated at 65° C. for 15 minutes, precipitated with ethanol, and resuspended in a volume of 20.5 μl. For ligation, two microliters of 100 μM of oligonucleotide-linker, 2.3 μl of 10× ligation buffer and 0.2 μl T4 DNA ligase (both from New England Biolabs, Beverly, Mass.) were added to the samples and the mixture incubated at room temperature for 6-16 hours. The oligonucleotide-linker is shown in FIG. 4A.

The ligated DNA fragments were purified using a Qiagen MinElute PCR purification Kit based on silica gel-membrane spin columns (Qiagen, Valencia, Calif.). Samples were processed in accordance with the manufacturer's instructions except that a 30% guanidine hydrochloride wash step was added prior to the regular wash. The product was eluted in 25 μl (15 μl+10 μl) elution buffer provided in the kit.

For PCR, 2.5 μl of the eluted DNA was amplified with AmpliTaq Gold® in 50 μl reaction with Gold buffer (Applied Biosystems, Foster City, Calif.), 100 μM dNTP, 1.2 M betaine, 1 μM Cyanine dye-labeled amplification primer (Cy-3 for the methylated mouse strain DNA and Cy-5 for the unmethylated mouse strain DNA) and 1 Unit of DNA polymerase. The primer sequence was as follows:

| | |
|---|---|
| Dye-5′-CTGACGATGAGTCCTGAGTCGG-3′ | (SEQ ID NO:6) |

The cycle condition was 95° C. for 6.5 minutes followed by 35 cycles of 94° C. for 30 sec, 55° C. for 20 seconds, and 72° C. for 1 minute. The amplified products (i.e., amplicons) were purified with the Qiagen PCR Purification kit and eluted in 10 μl of elution buffer.

A second PCR reaction using transgene primers, followed by agarose gel electrophoresis, was performed to determine if the amplicon contained the expected fragment predicted by the assay design. The result in FIG. 5B shows the expected strong amplification of transgene DNA fragment (see arrow) from the processed DNA of the unmethylated transgene mouse strain (lane D) while no amplification of a transgene DNA fragment was seen from the processed DNA of the methylated transgene mouse strain (lane B). The presence of a Cy-3 or Cy-5 label on the DNA amplicon did not affect the results.

Microarrays were prepared by spotting polynucleotides in triplicate onto a glass slide using a mechanical arrayer (Affymetrix, Santa Clara, Calif.). The Cy-3 and Cy-5 labeled DNA amplicons were mixed together and hybridized to the array in 6×SSC at 55° C. for several hours. The washing conditions were 2×SSC and 0.03% SDS; 2×SSC; 1×SSC; 0.2×SSC, all at room temperature. Arrays were scanned at 635 nm for the Cy-5 and 532 nm for Cy-3.

As shown in FIG. 5C, the oligonucleotide probe for the transgene detected higher Cy5 (from strain D) hybridization signal than Cy3 (from strain B) signal consistently in the triplet, indicating the successful detection of the methylation difference for the transgene. The control probe, as expected, detected a similar level of hybridization signal in both Cy5 and Cy3 channels consistent with the lack of methylation in the control gene in the two strains.

### Example 2

### Screening of Differentially Methylated Sites Using a cDNA Microarray

This example demonstrates the use of an embodiment of the present invention for detecting the extent of methylation of mouse genes using a high-density cDNA microarray. The mixture of Cy-3 and Cy-5 labeled DNA amplicons were prepared as described in Example 1 and hybridized to the M9K mouse cDNA microarray, which contains approximately 9000 sequence-verified cDNA clones spotted on a microscope slide. The hybridized array was scanned at 532nm and 635 nm as described in Example 1.

An overlay of the two scans was achieved by pseudo-coloring (green for Cy-3 and red for Cy-5). Four independent hybridizations were performed and the results indicate a high degree of reproducibility. Most of the spots show a yellow color of weak intensity, indicating that the genomic regions for the clones are not differentially methylated (they have similar methylation level) and likely are unmethylated in both samples. Red or green spots were observed for about 50 clones which have a Cy5/Cy3 ratio larger than 3 or smaller than 0.3, indicating a different level of methylation in the genomic regions they represent. Many spots show very weak or no signal, indicating that the regions detected by the immobilized cDNA are either highly methylated in both samples or are not represented in the amplicon (the latter may be due to the fact that the cDNA microarray contained many clones from the 3′ part of the genes that may not have CpG dinucleotides).

### Example 3

### Screening of Differentially Methylated Sites in Human Breast Cancers Using a 762-Feature CpG Island Fragment Microarray

In this example, 2 μg of genomic DNA from three human breast tumor cell lines, T47D, MB435, and SKBR3, were digested with HpaII overnight at 37° C. One fourth of the digested DNA was ligated to 10 μl of 100 μM of the unphosphorylated linker shown in FIG. 2 in 30 μl reaction at 16° C. for 5-16 hours. A 0.2 ml PCR tube contained the following mixture: 10 μl of the ligation product, 10 μl of 10× reaction buffer, 10 μl of 2 mM dNTPs, 20 μl of 5M betaine (Sigma-Aldrich, St Louis, Mo.), 5 μl DMSO, 44.5 μl H2O, and 0.5 μl (2.5 units) of Taq polymerase (Applied Biosystems, Foster City, Calif.). PCR conditions were as follows: 72° C., 5 minutes, then 94° C., 3 minutes followed by 25 cycles of 94° C., 30 seconds; 60° C., 30 seconds; 72° C., 3 minutes. The amplified samples were stored at 4° C. Amplified products (amplicons) were purified with Qiagen PCR purification kit (Qiagen, Valencia, Calif.). 2 μg of each amplicon was labeled either with Cy3 (for T47D) or Cy5 (for MB435 and SKBR3) dCTP to generate labeled probes as follows: 2 μg of amplicon (diluted in 21 μl with H₂O) and 20 μl of 2.5× random primer/reaction buffer (125 mM Tris pH 6.8, 12.5 mM MgCl2, 25 mM 2-mercaptoethanol, 750 μg/ml random octamers) are combined in a 0.5 ml eppendorf tube, incubated in boiling water for 5 minutes, placed on ice for 5 minutes. The samples were centrifuged briefly and the liquid fraction placed on ice, and the following was added: 5 μl 10× dNTP mix (1.2 mM each of dATP, dGTP and dTTP, 0.6 mM dCTP, 10 mM Tris pH8.0, 1 mM EDTA); 3 μl Cy5 or Cy3 dCTP (Amersham Pharmacia, 1 mM), and 1 μl klenow fragment (New England Biolabs). The mixture was incubated at 37° C. for 1.5 hours. The labeled probes were purified with a Microcon 30 filter (Amicon/Millipore).

The purified probes were mixed to form two mixtures: T47D/Cy3 with MB435/Cy5 and T47D/Cy3 with SKBR3/Cy5. The microarray composing of 762 CpG island fragments was manufactured as described in Example 1. The dye-labeled mixtures were hybridized to the microarray in a 65° C. water bath for 16 hours and washed and scanned as described (Example 1).

The results showed 25 CpG island fragments with significant differential hybridization signals (a ratio of Cy5/Cy3 greater than 3 or smaller than 0.3) indicating differential methylation between the cell lines. Bisulfite-based quantitative methylation assay COBRA (Xiong & Laird, Nucleic Acids Res. Jun. 15, 1997;25(12):2532-2534) was used to verify the detected differences in methylation among the samples. The COBRA assays confirmed differential methylation for six of nine tested CpG islands fragments.

The invention thus has been disclosed broadly and illustrated in reference to representative embodiments described above. Those skilled in the art will recognize that various modifications can be made to the present invention without departing from the spirit and scope thereof.

What is claimed is:

1. A method for detecting whether the extent of methylation of one or more regions of DNA in a test sample is different from that of a control, comprising:
generating DNA fragments from al least one test sample of DNA by cleaving methylation sites in the DNA that are not inethylatped while sparing methylation sites in the DNA that are methylated;
ligating an oligonucleotide-linker to the ends; of the DNA fragments;
amplifying the DNA fragments by initiating DNA amplification at the oligonucleotide linker with a linker-primer;
hybridizing the amplified DNA to one or more polynucleotides immobilized on a solid support, said polynucleotides being complementary to one or more regions of DNA in the test sample; and
comparing the amount of amplified DNA from the test sample that hybridizes to the immobilized polynucleotides versus that of a control, thereby detecting whether the extent of methylation of the one or more regions of DNA is different between the test sample and the control.

2. The method of claim 1, wherein said control is one or more control samples of DNA processed the same as the one or more test samples of DNA.

3. The method of claim 1, wherein said DNA fragments are generated by cleaving with a methylation-sensitive agent.

4. The method of claim 3, wherein said methylation-sensitive agent is a methylation sensitive restriction enzyme.

5. The method of claim 4, wherein said restriction enzyme cleaves a recognition sequence selected from the group consisting of CCGG, CCGC, GCGC, ACGT, CGGCCG, GCCGGC, GGCGCC, CCCGGG, CGCG, ATCGTA, TTCGAA, GTCGAG, and CTCGAG.

6. The method of claim 4, wherein said methylation-sensitive restriction enzyme cleaves a recognition sequence selected from the group consisting of: CGGCCG, GCCGGC, GGCGCC, CCCGGG, CCGG, CCGC, GCGC, and ACGT.

7. The method of claim 1 wherein said oligonucleotide linker is phosphorylated.

8. The method of claim 1 wherein said oligonucleotide linker is not phosphorylated.

9. The method of claim 8 wherein said non phosphorylated oligonucleotide linker is double stranded and wherein only one strand of the linker is ligated to the fragment.

10. The method of claim 9 wherein a polymerase chain mediated extension of the ligated fragment precedes the step of amplification and wherein one chain of the oligonucleotide linker is the linker primer.

11. The method of claim 1, further comprising the step of cleaving the DNA with at least one methylation-insensitive agent.

12. The method of claim 11, wherein said methylation-insensitive agent is a methylation-insensitive restriction enzyme.

13. The method of claim 12, wherein said methylation insensitive restriction enzyme is selected from the group consisting of EcoRI, ApoI, Tsp509I, MseI, BfaI, Csp6I, NlaIII, DpnII, and CviJI.

14. The method of claim 11, wherein said cleavage by the methylation-sensitive agent and the methylation-insensitive agent occurs simultaneously in the same mixture.

15. The method of claim 11, further comprising the step of ligating a non-phosphorylated oligonucleotide-linker to the end of each DNA fragment generated by the at least one methylation-insensitive agent.

16. The method of claim 15, wherein said oligonucleotide-linkers that ligate to sites cleaved by the methylation-insensitive agent are distinct from the oligonucleotide-linkers that ligate to the sites cleaved by the methylation-sensitive agent.

17. The method of claim 1, wherein said oligonucleotide-linker is a universal oligonucleotide-linker.

18. The method of claim 1, wherein said step of comparing the amount of amplified DNA that hybridizes is performed by measuring a detectable moiety associated with the amplified DNA.

19. The method of claim 18, wherein said detectable moiety is a cyanine dye.

20. The method of claims 18, wherein said detectable moiety is a labeled dendrimer.

21. The method of claim 18, wherein said detectable moiety is associated with the amplification primer prior to its incorporation into amplified product.

22. The method of claims 18, wherein said detectable moiety is attached to the amplified DNA following amplification.

23. The method of claim 18, wherein said detectable moiety is incorporated into the synthesized DNA during amplification.

24. The method of claim 1, wherein said immobilized polynucleotides comprise sequences complementary to one or more CpG islands.

25. The method of claim 1, wherein said immobilized polynucleotides comprise sequences complementary to one or more gene regulatory sequences or an encoding gene sequence.

26. The method of claim 1, wherein said immobilized polynucleotides comprise at least two polynucleotides immobilized as an array.

27. The method of claim 26, wherein said array is a microarray.

28. The method of claim 1, wherein said step of amplifying DNA occurs via the polymerase chain reaction.

29. The method of claim 2, wherein said DNA fragments from the test and control samples are labeled with different detectable moieties that are distinguishable from each other when mixed together.

30. The method of claim 29, wherein said different detectable moieties comprise a cyanine-3 dye and a cyanine-5 dye.

31. The method of claim 29, wherein said test and control samples are mixed together before the step of hybridization.

32. A method for detecting whether the extent of methylation of one or more regions of DNA in a test sample is different from that of a control, comprising:
generating DNA fragments from at least one test sample of DNA by cleaving methylation sites in the DNA that are not methylated while sparing methylation sites in the DNA that are methylated;
ligating an oligonucleotide-linker to the ends of the DNA fragments;
selecting a particular size range of the DNA fragments;
labeling the DNA fragments with a detectable moiety;
hybridizing the DNA fragments to one or more polynucleotides immobilized on a solid support, said polynucleotides being complementary to one or more regions of DNA in the test sample; and
comparing the amount of the detectable moiety associated with the immobilized polynucleotides for the test sample versus a control, thereby detecting whether the extent of methylation of the one or more regions of DNA is different between the test sample and the control.

33. The method of claim 32, wherein said control is one or more control samples of DNA processed the same as the one or more test samples of DNA.

34. The method of claim 32, wherein said DNA fragments are generated by cleaving with a methylation-sensitive agent.

35. The method of claim 34, wherein said methylation-sensitive agent is a methylation sensitive restriction enzyme.

36. The method of claim 35, wherein said restriction enzyme cleaves a recognition sequence selected from the group consisting of CCGG, CCGC, GCGC, ACGT, CGGCCG, GCCGGC, GGCGCC, CCCGGG, CGCG, ATCGTA, TTCGAA, GTCGAG, and CTCGAG.

37. The method of claim 35, wherein said methylation-sensitive restriction enzyme cleaves a recognition sequence selected from the group consisting of: CGGCCG, GCCGGC, GGCGCC, CCCGGG, CCGG, CCGC, GCGC, and ACGT.

38. The method of claims 32, wherein said detectable moiety is a labeled dendrimer.

39. The method of claim 32, wherein said one or more immobilized polynucleotides comprise sequences complementary to a one or more CpG islands.

40. The method of claim 32, wherein said one or more immobilized polynucleotides comprise sequences complementary to one or more gene regulatory sequences or an encoding gene sequence.

41. The method of claim 32, wherein said one or more immobilized polynucleotides comprise at least two polynucleotides immobilized as an array.

42. The method of claim 41, wherein said array is a microarray.

43. The method of claim 33, wherein said DNA fragments from the test and control samples are labeled with different detectable moieties that are distinguishable from each other when mixed together.

44. The method of claim 43, wherein said different detectable moieties comprise a cyanine-3 dye and a cyanine-5 dye.

45. The method of claim 43, wherein said test and control samples are mixed together before the step of hybridization.

46. The method of claim 32, wherein said DNA is labeled by labeling the oligonucleotide-linker.

47. The method of claim 46, wherein said oligonucleotide-linker comprises a capture sequence used for attaching the detectable label.

48. The method of claim 32, wherein said step of selecting a particular size range of the DNA fragments is conducted after the step of linker ligation.

49. The method of claim 32, wherein said step of selecting a particular size range of the DNA fragments step results in the separation of fragments about 1,500 base pairs in length or less.

50. A method of detecting whether an individual has a disease state associated with an abnormal extent of methylation of one or more regions of DNA in the individual, said method comprising:
providing a test sample of DNA derived from tissue of the individual;
determining the extent of methylation for one or more regions of DNA in the sample from the individual in accordance with the method of claim 1; and
determining abnormal methylation by relating the extent of methylation of the one or more regions of DNA in the test sample to that of a normal healthy control, thereby indicating if the individual has the state of disease.

51. A method of detecting whether an individual has a disease state associated with an abnormal extent of methylation of one or more regions of DNA in the individual, said method comprising:
providing a test sample of DNA derived from tissue of the individual;
determining the extent of methylation for one or more regions of DNA in the DNA of sample from the individual in accordance with the method of claim 32; and
determining abnormal methylation by relating the extent of methylation of the one or more regions of DNA in the test sample to that of a normal healthy control, thereby indicating if the individual has the state of disease.

52. A method of establishing whether the extent of methylation in a DNA region of an individual correlates with a disease state, said method comprising:
determining the extent of methylation for said region in the DNA for various individuals with the disease state and individuals without the disease state in accordance with the method of claim 1 and then determining if the extent of methylation of the region correlates with the disease state.

53. A method of establishing whether the extent of methylation in a DNA region of an individual correlates with a disease state, said method comprising:
determining the extent of methylation for said region in the DNA for various individuals with the disease state and individuals without the disease state in accordance with the method of claim 32 and then determining if the extent of methylation of the region correlates with the disease state.
